(19) United States
(12) Patent Application Publication
(10) Pub. No.: US 2002/0187067 A1
(43) Pub. Date: Dec. 12, 2002

(54) Adjustable accumulator

(76) Inventors: Robert G. Lukasik, Lake Elsinore, CA (US); Szu-Min Lin, Laguna Hills, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 09/864,470

(22) Filed: May 24, 2001

(51) Int. Cl. A61L 9/00; A62B 7/08; B01J 10/00; A61L 2/00

(52) U.S. Cl. 422/28; 422/32; 422/33; 422/125; 422/188; 422/236; 422/299; 422/300; 422/305

(57) ABSTRACT

An adjustable accumulator for delivering gases, liquids, or solids from a reservoir comprises an inner tube and a outer tube. One tube fits into the other tube to be sealingly engaged and to create a chamber in the interior. The tubes can move relative to each other to vary the space of the interior chamber. Another embodiment of the adjustable accumulator comprises the outer tube having a drum having a plurality of partitions. The drum partitions separate the drum into drum compartments. The drum partitions fit tightly against the inner tube. The drum compartments are in fluid communication with the interior of the inner tube when the inner tube is positioned such that a bottom edge of the upper tube is aligned with any drum partition that is at a higher level than the drum compartment or is between two drum partitions which form the drum compartment. The adjustable accumulator can be used to deliver vaporizable germicides to a sterilization chamber. A single adjustable accumulator can be used to deliver various amounts of vaporizable germicide to different sizes of sterilization chamber by adjusting the cumulative volume of the central chamber and outside compartments.

### BACKGROUND OF THE INVENTION

1 Field of the Invention

This invention relates to an adjustable accumulator for delivering gas, liquid, or solids, for use, for example, in sterilization.

2 Description of the Related Art

Sterilization is used in a broad range of industrial and medical applications. Sterilization is the complete destruction or the irreversible inactivation of all the microorganisms in the sterilizer. Sterilization can be performed, for example, with heat or chemical treatment. Heat sterilization is normally done using steam. Some equipment cannot withstand the heat or the moisture of steam treatment. As a result, chemical sterilization is now commonly used.

Chemical disinfection or sterilization can be done using alcohols, aldehydes such as formaldehyde, phenols, ozone, ethylene oxide, chlorine dioxide, peracetic acid, or hydrogen peroxide. Hydrogen peroxide and ethylene oxide are commonly used for chemical sterilization.

U.S. Pat. No. 4,653,876, incorporated herein by reference, discloses an exemplary sterilization process in which a vaporizable germicide such as hydrogen peroxide is introduced into an evacuated sterilization chamber. The vaporizable germicide vaporizes and is allowed to diffuse throughout the chamber and onto the items to be sterilized. After a period of time, electrical energy is applied to an electrode to form a plasma to complete the sterilization cycle.

The STERRAD® Sterilization System is an exemplary hydrogen peroxide sterilization system utilizing low-temperature plasma, commercially available from Advanced Sterilization Products, Irvine, Calif. The system employs an automated delivery system in which a measured amount of the liquid germicide, typically aqueous hydrogen peroxide, is delivered to the sterilization chamber. Measured portions of the liquid germicide are provided in rupturable cells within a liquid cassette housing. The cassette and the delivery system are fully described in the Williams et al. patents, U.S. Pat. Nos. 4,817,800, issued Apr. 4, 1989; 4,913,196, issued Apr. 3, 1990; 4,938,262, issued Jul. 3, 1990; and 4,941,518, issued Jul. 17, 1990, all of which are incorporated herein by reference in their entirety.

With the nature of the art, many and/or large pieces of equipment are required to be sterilized in a short period of time; therefore, large sterilization chambers are needed to accommodate such volumes. Although the cassette and the delivery system are adequate for most applications, the volumes of vaporizable germicide that can be delivered to the sterilization chamber with the cassette delivery system are limited to incremental volumes of single cells on the cassette. Various sizes of large sterilization chambers require an administration of large amounts of vaporizable germicide. Delivering large volumes of sterilant would require a large number of cassettes. There is a need for a single piece of apparatus to deliver vaporizable germicide to large chambers of different sizes all at one time. Additionally, with the administration of large volumes of liquid samples, the bulkiness of the large volume of liquid sample may add to the hazard of handling for the operator. Because of the inherent toxicity of certain germicides, there is a need for a delivery system to the sterilization chambers that is easy to handle.

U.S. Pat. No. 4,744,955 discloses an adjustable volume pipette sampler in which the volume of delivered liquid is controlled by a pair of barrels that are axially aligned, retained together by complementary screw threads, and adjustable by relative rotation of the barrels.

While the adjustable volume pipette sampler offers a means to deliver varying volumes of liquid samples, the sampler is hard to manipulate, especially with large volumes of liquid samples. To adjust between large volumes requires a great deal of time and energy of the operator. Therefore, there is a need for a simple, safe, and quick system to deliver large amounts of liquid samples.

There is a need for a simple, safe, precise, and inexpensive system for introducing vaporizable germicide into a sterilization chamber in which the amount of vaporizable germicide can be varied. There is a need for a simple vaporizable germicide delivery system that can deliver a wide range of volumes, including very large volumes, of vaporizable germicide to match the needs of various sizes of sterilization chambers or rooms.

### SUMMARY OF THE INVENTION

One aspect of the invention involves an adjustable accumulator for delivering a chemical, which can be a gas, liquid, or solid. The adjustable accumulator includes a hollow outer tube having a first open end and a second open end connected to a first valve; and a hollow inner tube having a first open end and a second end connected to a second valve. The first open end of the inner tube is adapted to movably and securely connected to the first open end of the outer tube so as to retain the chemical within the volume, with the volume being the space enclosed within the outer tube, the inner tube, the first valve, and the second valve. Preferably, the outer tube and the inner tube are cylindrical. The adjustable accumulator can include at least one O-ring. To hold the inner tube and outer tube in a desired position, a retainer can be used. Examples of retainers include setscrews, spacers, and shafts or jacks.

In an embodiment of the invention, the adjustable accumulator has an outer tube which has at least one drum. The drum has an interior, such that the volume of retainment of the chemical includes the interior when the inner tube is adjusted to a position so that the interior of the drum is in fluid communication with the volume. The drum can be oval, circular, or any polygonal shape and does not necessarily surround the entire outer tube.

In another embodiment of the invention, the adjustable accumulator has an outer tube which has a drum that has at least one partition to separate the interior of the drum into at least two compartments. The compartment has a compartment interior where the volume of retainment of the chemical includes the compartment interior when the inner tube is adjusted to a position so that the compartment interior is in fluid communication with the volume. The compartment can be detachable from the adjustable accumulator. The compartments can be substantially identical in size and shape. The partition can be downwardly sloped toward the inner tube.

Another aspect of the invention involves a system for equipment. The system includes an adjustable accumulator, a germicide reservoir to hold germicide, preferably hydrogen peroxide, and a sterilization chamber. The adjustable accumulator includes a hollow outer tube having a first open end and a second open end connected to a first valve; and a hollow inner tube having a first open end and a second end connected to a second valve. The germicide reservoir and the sterilization chamber are in fluid communication with the adjustable accumulator. The system can further include a vaporizer, vacuum pump, and/or source of plasma. The system can situate the first valve to be located between the adjustable accumulator and the sterilization chamber and the second valve to be located between the adjustable accumulator and the reservoir.

In other embodiments of the invention, the system can include the adjustable accumulator with the outer tube that has at least one drum. The drum can have at least one partition to separate the interior of the drum into at least two compartments.

Another aspect of the invention involves a method for sterilizing an article in a chamber. The method includes placing the article in the chamber; delivering a volume of germicide, preferably hydrogen peroxide, from a reservoir containing germicide to the chamber via the adjustable accumulator; and contacting the germicide with the article. The method can further include evacuating the chamber, vaporizing the germicide, opening and/or closing the second valve between the adjustable accumulator and the reservoir, opening and/or closing the first valve between the adjustable accumulator and the chamber, and/or moving the inner tube and/or outer tube so as to adjust the volume of the adjustable accumulator.

In other embodiments of the invention, the method can include the adjustable accumulator with the outer tube that has at least one drum. The drum can have at least one partition to separate the interior of the drum into at least two compartments.

### BRIEF DESCRIPTION OF THE DRAWINGS

FIG. 1 is a schematic drawing showing a sterilization system and a cross section of an adjustable accumulator according to an embodiment of the invention;

FIGS. 2A-C show adjustable accumulators comprising an upper tube and a lower tube;

FIGS. 3A-C show the relative positions of the upper tube and the lower tube of the adjustable accumulator of FIG. 2;

FIG. 4 is a schematic drawing showing an adjustable accumulator with an upper tube and a lower tube comprising a drum made up of a plurality of drum compartments that are separated by drum partitions, where a bottom edge of the upper tube is in contact with a bottom wall of the lower tube;

FIG. 5 is a schematic drawing showing the adjustable accumulator of FIG. 4, where a bottom edge of the upper tube is in contact with the first drum partition;

FIG. 6 shows an embodiment of an adjustable accumulator in which retainers that hold an upper tube and a lower tube in place comprise setscrews;

FIG. 7 shows an embodiment of an adjustable accumulator in which retainers that hold an upper tube and a lower tube in place comprise spacers;

FIG. 8 shows an embodiment of an adjustable accumulator in which retainers that hold an upper tube and a lower tube in place comprise a shaft or jack;

FIG. 9 shows a plurality of drums that extend from a lower tube and are separated from each other;

FIG. 10 shows a plurality of drum compartments that extend from a lower tube and are connected to each other;

FIG. 11 shows a lower tube that consists of a drum with partitions;

FIG. 12 shows a plurality of drum compartments that extend from a lower tube and are of different sizes;

FIG. 13 is a schematic drawing of a top-view of an adjustable accumulator with a drum having a circular shape;

FIG. 14 is a schematic drawing of a top-view of an adjustable accumulator with a drum having an oval shape;

FIG. 15 is a schematic drawing of a top-view of an adjustable accumulator with a drum having a square shape;

FIG. 16 is a schematic drawing of a top-view of an adjustable accumulator with a drum having a polygonal shape;

FIG. 17 shows an embodiment of a drum of an adjustable accumulator, where the contact point between adjacent detachable drum compartments is a bottom wall of a drum compartment and a top wall of the adjacent drum compartment;

FIG. 18 shows an embodiment of a drum of an adjustable accumulator, wherein the contact point between detachable adjacent drum compartments is a side wall of a drum compartment and a bottom wall of an adjacent drum compartment;

FIG. 19 shows an embodiment of a drum of an adjustable accumulator, wherein the contact point between adjacent detachable drum compartments is a side wall of a drum compartment and a top wall of an adjacent drum compartment;

FIG. 20 shows an arrangement of an O-ring to form a seal between an upper tube and a lower tube of an adjustable accumulator in which the O-ring is located on the inside of the outer tube;

FIG. 21 shows an arrangement of an O-ring to form a seal between an upper tube and a lower tube of an adjustable accumulator in which the O-ring is located on the outside of the inner tube;

FIG. 22 shows an arrangement of O-rings to form a seal between an upper tube and a lower tube of an adjustable accumulator in which the O-rings are located on the inside of the outer tube and on the outside of the inner tube;

FIG. 23 shows an embodiment of a drum of an adjustable accumulator, wherein a drum partition includes an O-ring at the contact point with the upper tube;

FIG. 24 shows an embodiment of an adjustable accumulator mounted with a reservoir and a vaporizer in which the transfer of vaporizable germicide between the components proceeds via gravity, pressure, or vacuum;

FIG. 25 shows an embodiment of an adjustable accumulator in connection with a reservoir and a vaporizer via tubes in which the transfer of vaporizable germicide between the components proceeds via a vacuum;

FIG. 26 is a schematic drawing of the sterilization system and the adjustable accumulator after a vaporizable germicide has been admitted into the upper tube of the adjustable accumulator;

FIG. 27 is a schematic drawing of the sterilization system and the adjustable accumulator after a bottom edge of the upper tube has been raised and placing one drum compartment in fluid communication with the upper tube, allowing a vaporizable germicide in the upper tube to enter one drum compartment; and

FIG. 28 is a schematic drawing of the sterilization system and the adjustable accumulator after a bottom edge of the upper tube has been raised and placing two drum compartments in fluid communication with the upper tube, allowing a vaporizable germicide in the upper tube to enter the two drum compartments.

Reference Numerals in Drawings

| | | | |
|---|---|---|---|
| 10 | sterilization chamber | 100 | top edge |
| 20 | adjustable accumulator | 110 | side wall |
| 24 | reservoir | 120 | bottom wall |
| 26 | vaporizable germicide | 130 | drum |
| 28 | vaporizer | 140 | drum bottom |
| 30 | first valve | 150 | drum side |
| 32 | second valve | 160 | drum top |
| 36 | vacuum pump | 170 | drum partition |
| 40 | valve | 180 | drum compartment |
| 50 | upper tube | 190 | setscrew |
| 60 | lower tube | 200 | notch |
| 70 | bottom edge | 210 | spacer |
| 80 | side wall | 220 | shaft or jack |
| 90 | top wall | 230 | O-ring |

### DETAILED DESCRIPTION OF THE PREFERRED EMBODIMENT

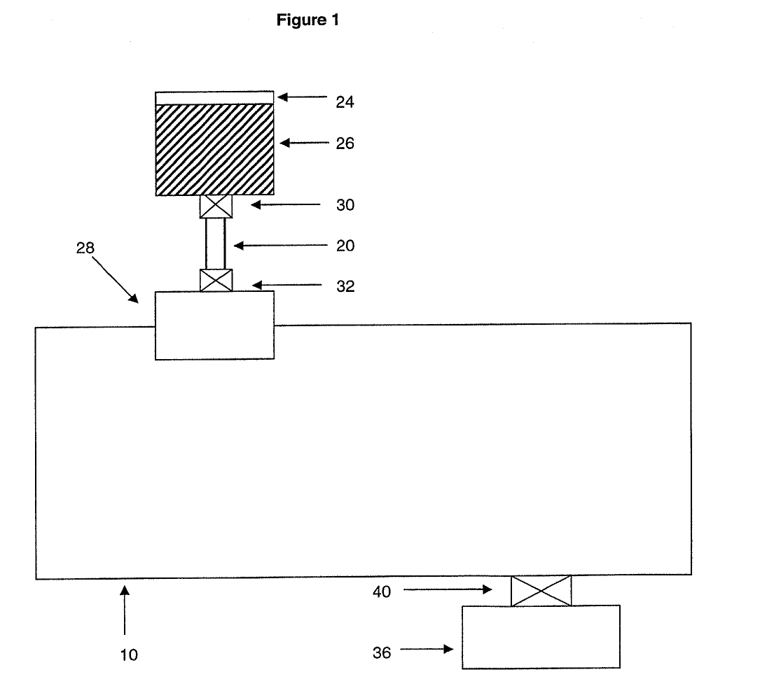

FIG. 1 is a schematic diagram of a sterilization chamber 10 with an adjustable accumulator 20 according to one embodiment of the invention. The sterilization chamber 10 and its components and methods of use are described in detail in U.S. Pat. No. 4,756,882, issued Jul. 12, 1988; U.S. Pat. No. 5,656,238, issued Aug. 12, 1997; and U.S. Pat. No. 6,060,019, issued May 9, 2000, all of which are incorporated herein by reference in their entirety. The adjustable accumulator 20 is mounted between a reservoir 24 that contains a vaporizable germicide 26, and a vaporizer 28, which is located above and is in fluid communication with the sterilization chamber 10. The term “germicide” as used herein includes both sterilants and disinfectants. First and second valves 30 and 32 are located between the reservoir 24 and the adjustable accumulator 20 and between the adjustable accumulator 20 and the vaporizer 28. A vacuum pump 36 and a valve 40 are fluidly connected with the sterilization chamber 10.

The accumulator can be designed to be adjustable to have the capability to deliver different volumes of materials. Although the adjustable accumulator 20 is described in the context of an adjustable accumulator for delivering vaporizable germicide 26 to a sterilization chamber or room 10, it is to be understood that the application of the adjustable accumulator 20 to sterilization is illustrative only. The adjustable accumulator 20 of the present invention has many uses, and the example of delivering vaporizable germicide to a sterilization chamber 10 is exemplary and is not meant to be limiting. Further, the adjustable accumulator 20 can be used to deliver gases, liquids, solids, and slurries of solids in one or more liquids.

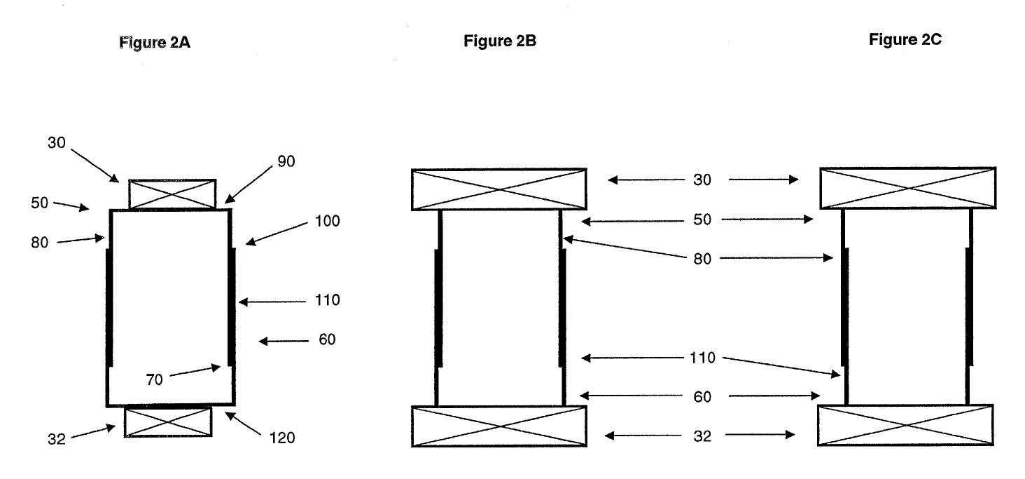

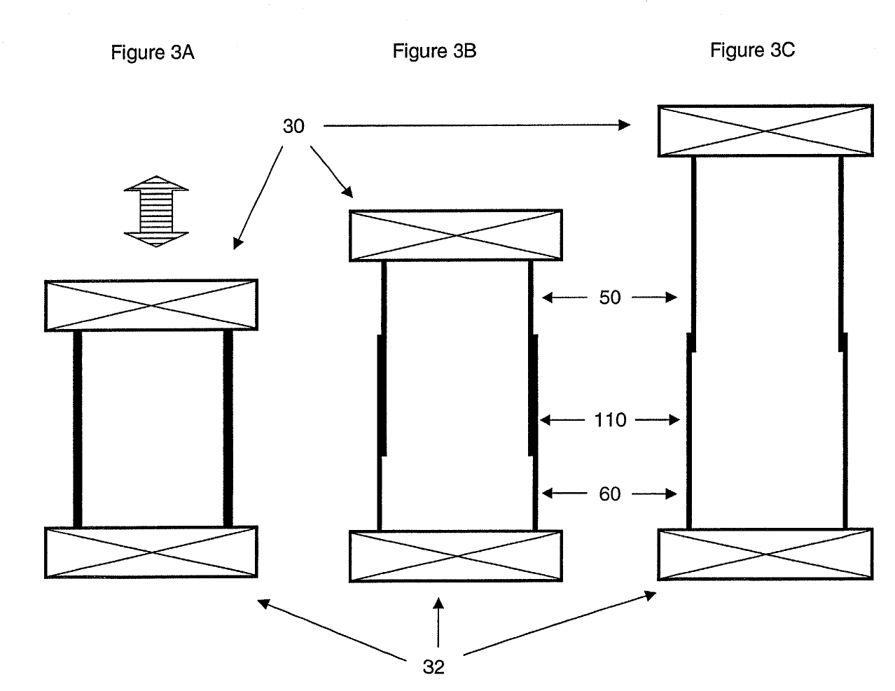

The invention will now be described with reference to FIGS. 2-3, which illustrate basic embodiments of the adjustable accumulator. FIGS. 2A-C are schematic diagrams of the adjustable accumulator, which comprises two parts: an upper tube 50 and a lower tube 60. The upper tube 50 comprises a bottom edge 70, a side wall 80, and a top wall 90 that is connected to the first valve 30. The lower tube 60 comprises a top edge 100, a side wall 110, and a bottom wall 120 that is connected to the second valve 32. The lengths of longitudinal axes of the upper tube 50 and the lower tube 60 can be of any length and are not necessarily equal to each other. One tube fits into the other tube to be sealingly engaged and to create a chamber in the interior. In the preferred embodiment of the invention, the upper tube 50 is the inner tube and the lower tube 60 is the outer tube that surrounds the upper tube 50 and is sealingly engaged with the upper tube 50. In another embodiment, the lower tube 60 is the inner tube and the upper tube 50 is the outer tube that surrounds the lower tube 60 and is sealingly engaged with the lower tube 60. In both instances, the inner tube is movable within the outer tube so that the inner tube can be placed in different positions relative to the outer tube; therefore, the volume of the chamber is adjustable. The tubes can take any shape, preferably circular, oval, or any polygonal shape, as long as the inner tube is sealingly engaged with the outer tube.

FIGS. 3A-C show the relative positions of the upper tube 50 and the lower tube 60 of the preferred embodiment in which the upper tube 50 is the inner tube and the lower tube 60 is the outer tube. Although FIGS. 3A-C describe the dynamic movement of one embodiment of the adjustable accumulator, the example is not meant to be limiting. For example, the description of the dynamic movement of the invention applies also to the situation where the upper tube 50 is the outer tube and the lower tube 60 is the inner tube. The adjustable accumulator is shown in the lowest position in FIG. 3A. In FIG. 3A, the length of the longitudinal axis of the chamber that is created by the upper tube 50 and the lower tube 60 is equal to about the length of the upper tube 50 or lower tube 60, whichever is longer. In FIG. 3B, the upper tube 50 and lower tube 60 have been shifted from the lowest position in FIG. 3A to alter the volume of the chamber. The upper tube 50 and lower tube 60 can hold any intermediate position where the side wall 80 of the upper tube 50 can overlap with the side wall 110 of the lower tube 60. As shown in FIG. 3C, the maximum length of the longitudinal axis of the chamber is equal to about the sum of the lengths of the longitudinal axes of the upper tube 50 and the lower tube 60.

The invention will now be described with reference to FIGS. 4 and 5, which illustrate other embodiments of the invention. The adjustable accumulator comprises compartments to accommodate additional volume. FIG. 4 is a schematic diagram of the adjustable accumulator 20 situated between the reservoir 24 that contains vaporizable germicide 26 and the vaporizer 28. The adjustable accumulator 20 of FIGS. 4 and 5 comprises the upper tube 50 and the lower tube 60, where the upper tube 50 fits in the lower tube 60 and is sealingly engaged with the lower tube 60. The lower tube 60 comprises a drum 130. The drum 130 may be situated anywhere along the longitudinal axis of the lower tube 60. Alternatively, the lower tube 60 consists of the drum 130. The drum 130 needs not necessarily to surround the entire upper tube 50. The drum comprises a drum bottom 140, a drum side 150, and a drum top 160. Situated in the interior of the lower tube 60, the upper tube 50 is preferably a vertically movable cylindrical tube whose length is equal to or exceeds the height of the lower tube 60 and has the ability to traverse the center of the lower tube 60. A plurality of drum partitions 170 that extend from the drum side 150 are able to make contact with the vertically movable upper tube 50, depending on the position of the upper tube 50. The drum 130 is divided into a plurality of drum compartments 180 by the plurality of drum partitions 170. The adjustable accumulator 20 is in fluid communication with the reservoir 24 and with the vaporizer 28 through gas-tight seals. The upper tube 50 communicates with the reservoir 24 through the first valve 30, located between the upper tube 50 and the reservoir 24. The lower tube 60 communicates with the vaporizer 28 through the second valve 32, located between the lower tube 60 and the vaporizer 28.

In FIG. 4, the upper tube 50 is positioned at the lowest position with the bottom edge 70 of the upper tube 50 in contact with the bottom wall 120 of the lower tube 60. The upper tube 50 traverses the lower tube 60. Alternatively, the lowest position of the adjustable accumulator 20 could position the bottom edge 70 of the upper tube 50 in contact with the drum bottom 140. When the upper tube 50 is in the lowest position, all drum partitions 170 fit tightly against the upper tube 50, forming a gas-tight seal with the upper tube 50. Because the bottom edge 70 of the upper tube 50 is in contact with the bottom wall 120 of the lower tube 60 or the drum bottom 140, none of the drum compartments 180 is in fluid communication with the interior of the upper tube 50.

In FIG. 5, the upper tube 50 has been raised such that the bottom edge 70 of the upper tube 50 makes contact and is sealingly engaged with the first drum partition 170, thereby placing the lowest drum compartment 180 into fluid communication with the interior of the upper tube 50. The interior of any particular drum compartment 180 is blocked from the upper tube 50 when the bottom edge 70 of the upper tube 50 is in contact with a drum partition 170 at a level lower than the particular drum compartment 180. The interior of any particular drum compartment 180 is exposed to the interior of the upper tube 50 when the bottom edge 70 of the upper tube 50 is in contact with a drum partition 170 at a level higher than the particular drum compartment 180. Moving the upper tube 50 up and down varies the number of drum partitions 170 that are in contact with the upper tube 50 and, therefore, the number of drum compartments that are in fluid communication with the interior of the upper tube 50. Under certain embodiments, the fluid communication between the drum compartment 180 and the interior of the upper tube 50 can also be achieved by resting the bottom edge 70 of the upper tube 70 in between two drum partitions 170.

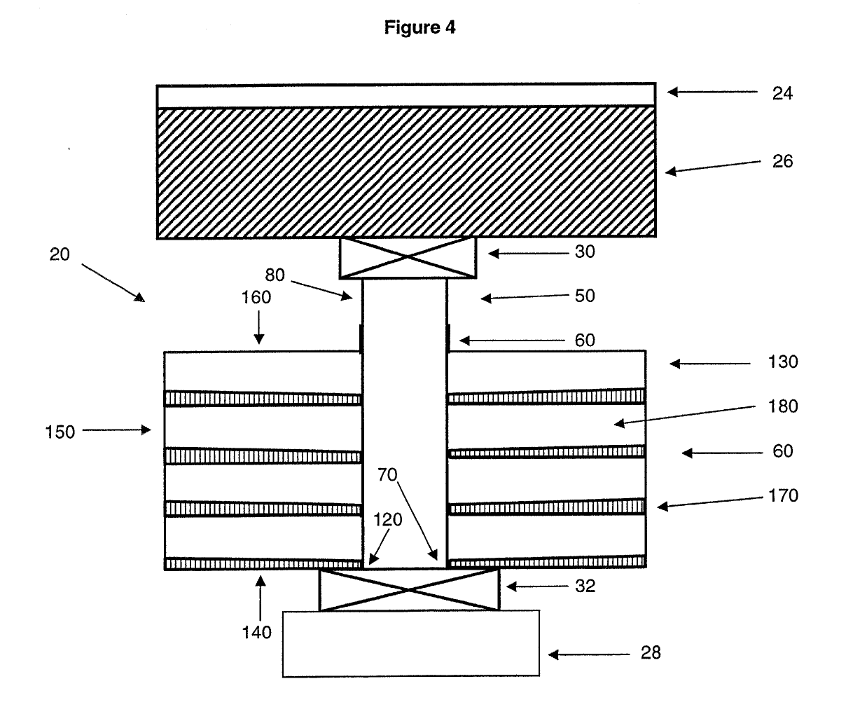

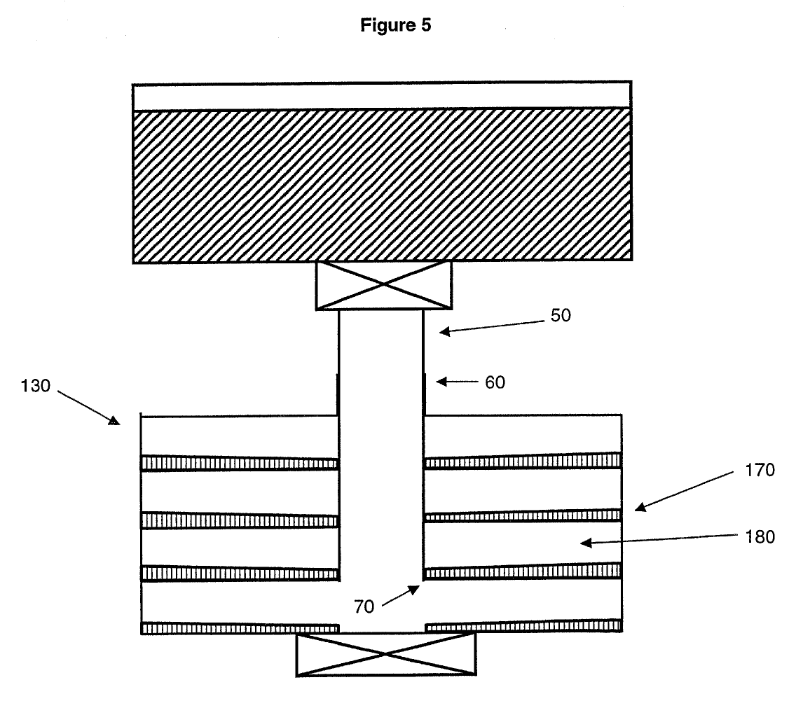

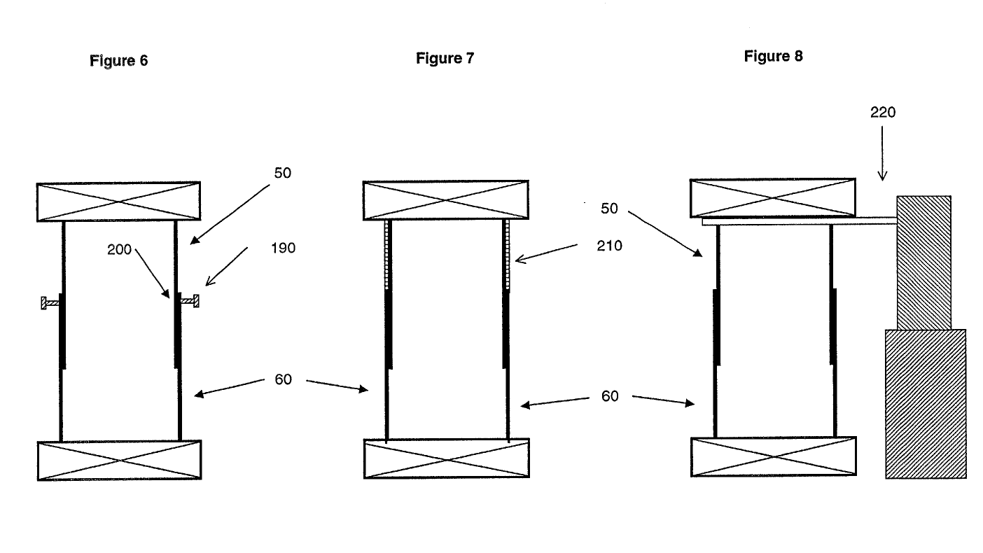

The vertically movable upper tube 50 and lower tube 60 can be held at various vertical levels by retainers. FIGS. 6-8 show embodiments displaying mechanisms to secure the upper tube 50 and lower tube 60 in place in which the upper tube 50 is the inner tube and the lower tube 60 is the outer tube, though the same mechanisms can work for other embodiments of the invention as well. FIG. 6 shows an embodiment in which a plurality of setscrews 190 fit through the lower tube 60 into a plurality of notches 200 of the upper tube 50 to hold the lower tube 60 and the upper tube 50 in place. The plurality of notches 200 are sized so as to fit with the plurality of setscrews 190. The notches 200 are located along different lengths of the longitudinal axis of the lower tube 60 on its outer diameter and do not extend to the inner diameter of the lower tube 60.

In FIG. 7, the retainer is a spacer 210 that fits exactly into a space along the side wall of the inner tube, whether the upper tube 50 or lower tube 60, that is exposed when the inner tube and outer tube are positioned in an intermediate position. The spacer 210 is placed between the edge of the outer tube and end of the inner tube next to the valve to prevent motion of the inner tube or outer tube. The spacer 210 can be of any length to accommodate various intermediate positions of the inner tube and the outer tube.

In FIG. 8, the retainer is a shaft or jack 220 with a side arm used to support the upper tube 50 by directly connecting to the upper tube 50. The shaft or jack 220 sits in a position to be able to hold the upper tube 50 in place. The shaft or jack 220 can be operated manually, hydraulically, pneumatically, mechanically, or electrically with a motor, such as a stepper motor. The apparatus is not limited by the use of the mechanisms mentioned as retainers; any device that can hold the upper tube 50 in place at various vertical levels is considered to be a suitable retainer.

Referring again to FIG. 1, the reservoir 24 and the adjustable accumulator 20 are in fluid communication when the first valve 30 is opened. Closing the first valve 30 prevents the vaporizable germicide 26 from entering the adjustable accumulator 20. The junction of the reservoir 24 and the adjustable accumulator 20 is preferably able to hold a gas-tight seal. The first valve 30 may comprise two pieces in which one piece may be directly affixed to the top of the upper tube 50 or to the bottom of the reservoir 24. Then, the adjoining piece of the first valve 30 on the opposing end preferably fits with the first portion of the first valve 30 as to create a gas-tight seal. The gas-tight seals will be discussed in detail below.

Referring again to FIG. 1, opening the second valve 32 allows fluid communication between the adjustable accumulator 20 and the vaporizer 28. Closing the second valve 32 prevents the vaporizable germicide 26 from entering the vaporizer 28. The junction of the adjustable accumulator 20 and the vaporizer 28 is preferably gas-tight. Specifically, the second valve 32 is located at the bottom of the lower tube 60. Like the first valve 30, the second valve 32 may comprise two pieces in which one piece may be directly affixed to the bottom of the lower tube 60 or to the top of the vaporizer 28. Then, the adjoining piece of the second valve 32 on the opposing end preferably fits with the first portion of the second valve 32 as to create a gas-tight seal. The gas-tight seals will be discussed in detail below.

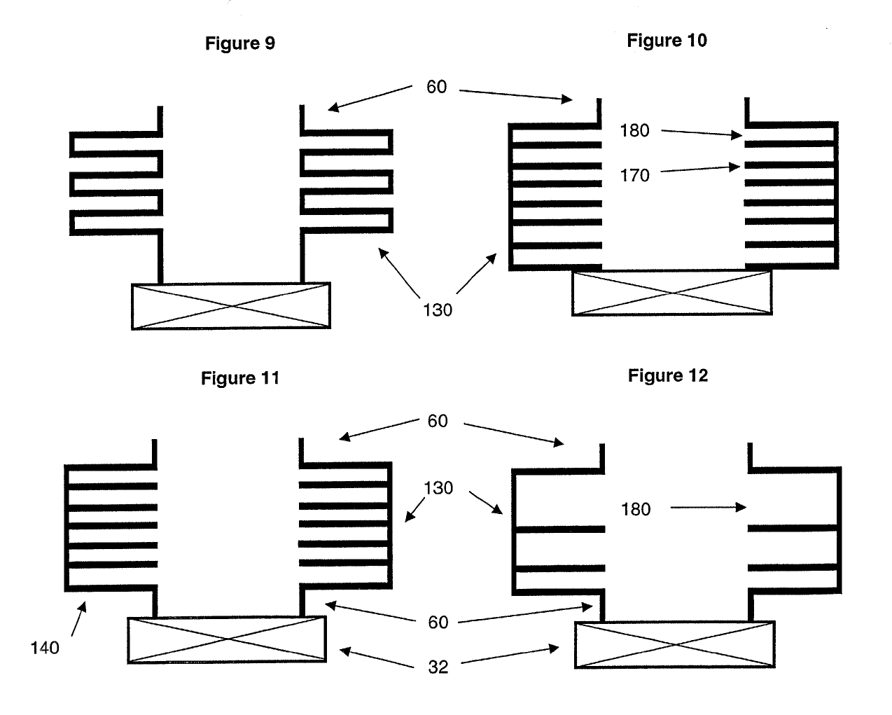

The drum 130 can extend from the lower tube 60 in several ways. FIGS. 9-12 show the lower tube 60 in several embodiments. FIG. 9 shows the plurality of drums 130 extending from the lower tube 60 where the drums 130 are separated from each other. FIG. 10 shows the drum 130 extending from the lower tube 60 where the drum compartments 180 are connected to each other and separated with drum partitions 170. FIG. 11 shows the plurality of drum compartments 180 extending from the lower tube 60 where the drum bottom 140 does not rest on the second valve 32. FIG. 12 shows the plurality of drum compartments 180 extending from the lower tube 60 where the drum compartments 180 are of different sizes.

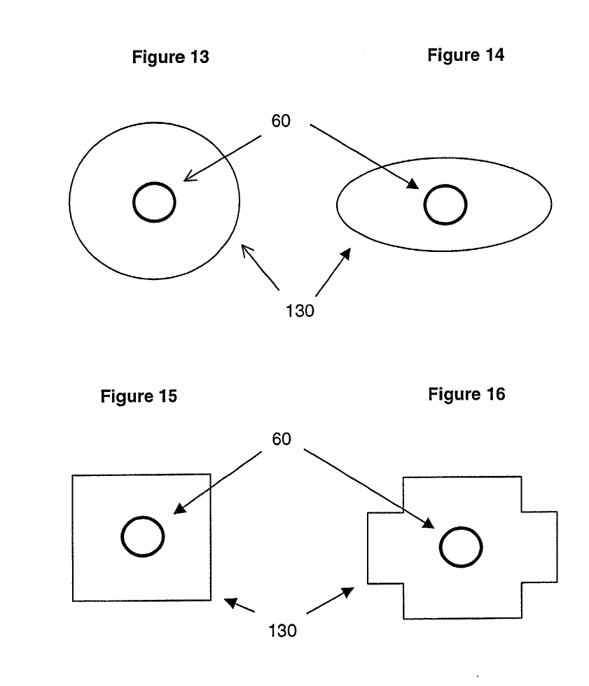

The upper tube 50, the lower tube 60, and the drum 130 can have any shape so as long as the upper tube 50 fits into and is movable within the lower tube 60. In exemplary embodiments, the shape of the lower tube 60 or drum 130 is circular, oval, square, or twelve-sided polygonal, as seen in FIGS. 13-16. For certain embodiments, the drum 130 may not surround the entire lower tube 60. The lower tube 60 and the drum 130 may vary in size so as to allow delivery of a wide range of volumes of the vaporizable germicide to match the needs of various sizes of sterilization chambers. In embodiments appropriate for sterilizers, the drum may have a volume up to about 100 liters, preferably about 1 to about 1000 milliliters, more preferable about 2 to about 100 milliliters.

There is no limit to the total number of drum compartments 180 that occupy the drum 130. In one embodiment, the drum compartments 180 are detachable from the adjustable accumulator 20. The horizontally extending drum compartments 180 are stacked one above another. In one embodiment, the drum compartments 180 detach one at a time from the adjustable accumulator 20. In another embodiment, more than one drum compartment 180 are fused together to from a single unit and are detached from the adjustable accumulator 20 as a unit.

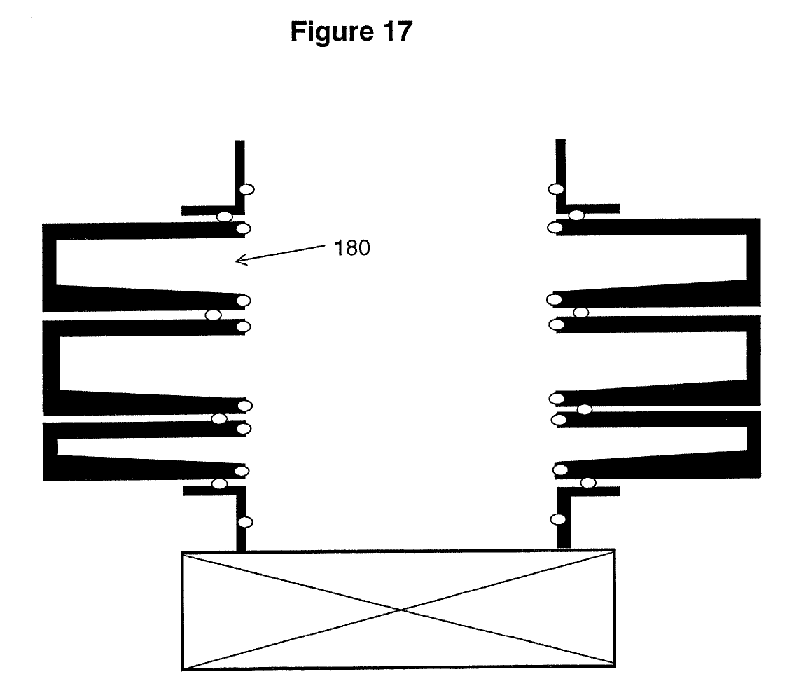

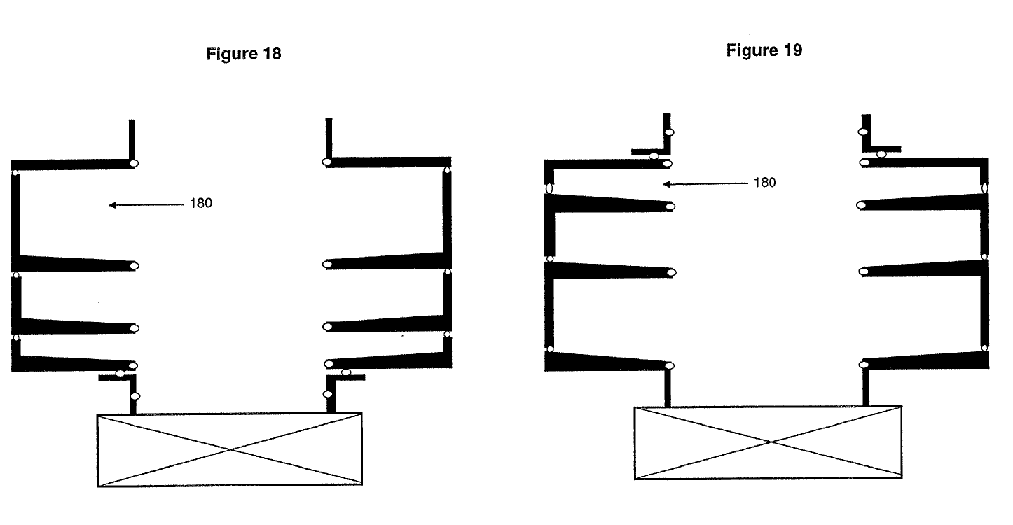

When the drum compartments 180 stack one on top of another and are detachable from one another, the contact points between the adjacent detachable drum compartments 180 may be oriented differently in different embodiments. Also, shown in FIGS. 17-19, the drum partitions 170 can be downwardly sloped to allow thorough delivery of germicide. FIG. 17 shows an embodiment in which the contact point between the adjacent detachable drum compartments 180 is a bottom wall of a drum compartment and a top wall of the adjacent drum compartment. FIG. 18 shows an embodiment in which the contact point between the adjacent detachable drum compartments 180 is a side wall of a drum compartment and a bottom wall of the adjacent drum compartment. FIG. 19 shows an embodiment in which the contact point between the adjacent detachable drum compartments 180 is a side wall of a drum compartment and a top wall of the adjacent drum compartment.

The adjustable accumulator 20 can be made from a wide range of materials, including metal, glass, or plastic. Suitable metals include steel or aluminum. Aluminum and TEFLON™ are exemplary materials for forming the adjustable accumulator 20. TEFLON™ is a tradename for polytetrafluoroethylene.

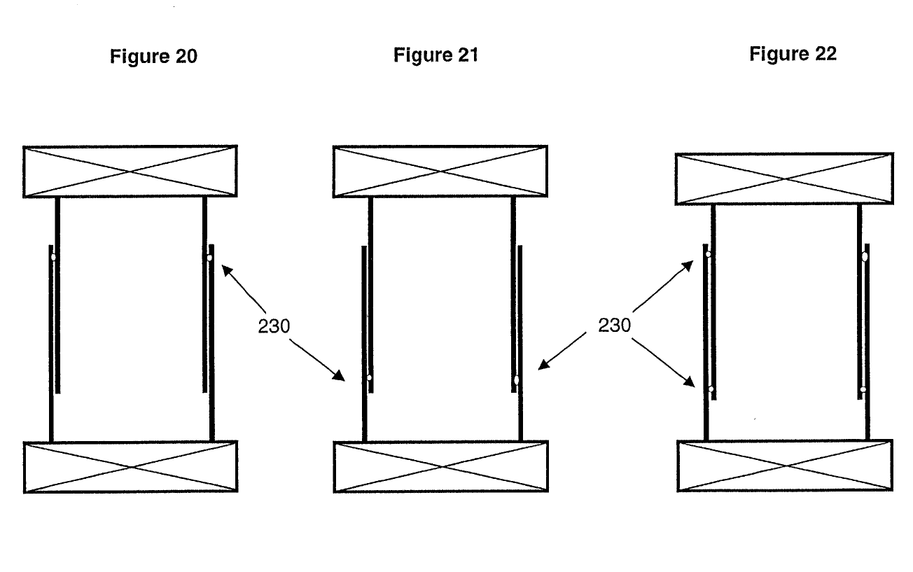

Preferably, the apparatus of the invention includes gas-tight seals in the adjustable accumulator 20 between the upper tube 50 and the lower tube 60. A seal between the bottom edge 70 of the upper tube 50 and the top edge 100 of the lower tube 60 can be achieved in several ways, depending on the material from which the adjustable accumulator 20 is fabricated. In one embodiment, both the upper tube 50 and the lower tube 60 are made of TEFLON™. In another embodiment, one of the tubes is made of TEFLON™ and the other tube is made of metal. In another embodiment, one of the tubes is made of TEFLON™ and the other tube is made of glass. If the upper tube 50 and the lower tube 60 are properly fabricated, the contact between the upper tube 50 and the lower tube forms a seal. In another embodiment, both the upper tube 50 and the lower tube 60 are made of metal. As shown in FIGS. 20-22, O-rings 230 or packing can be placed on the upper tube 50 and/or the lower tube 60 in different arrangements to form a seal. The O-rings 230 can be placed on the inside of the outer tube, as shown in FIG. 20, or on the outside of the inner tube, as shown in FIG. 21, or on both the inside of the outer tube and the outside of the inner tube, as shown in FIG. 22.

If O-rings 230 or packing are used in the adjustable accumulator 20, the O-rings 230 or packing are preferably formed of a material that is resistant to the vaporizable germicide 26 that is used. VITON™ is an exemplary material for forming the O-rings 230 or packing. TEFLON™ or silicone may also be used to form the O-rings 230 or packing.

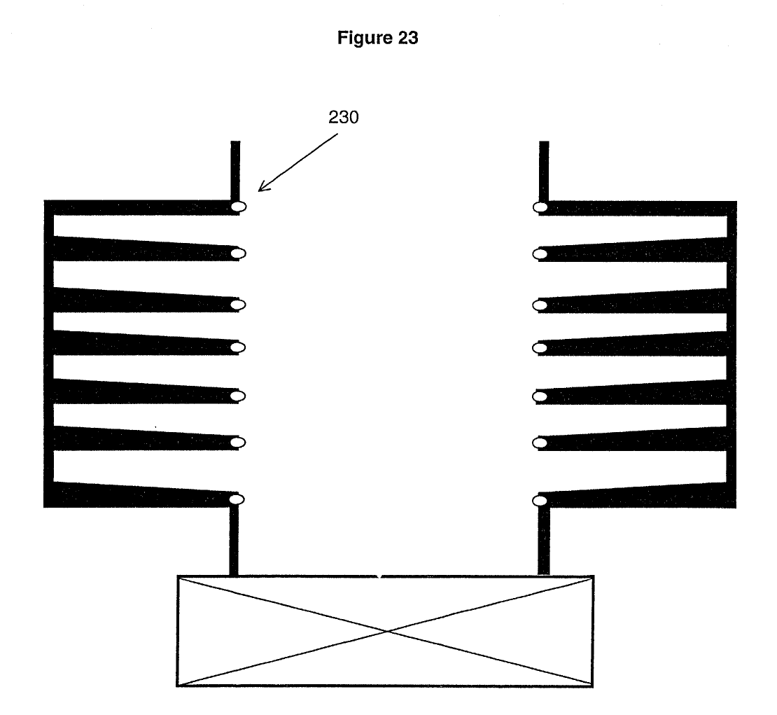

Likewise, in the case of the adjustable accumulator 20 of FIG. 4 which has the drum 130, the apparatus of the invention includes gas-tight seals in the adjustable accumulator 20 between the side wall 80 of the upper tube 50 and the drum partition 170. If the upper tube 50 is positioned in the lowest position as shown in FIG. 4, then the bottom edge 70 of the upper tube 50 is aligned with the lowest drum partition 170 or the bottom wall 120 of the lower tube 60. A seal between the bottom edge 70 of the upper tube 50 and the drum partition 170 can be achieved in the same manner as that of the seals between the upper tube 50 and the lower tube 60 mentioned previously. If O-rings 230 are to be used, FIG. 23 shows one possible arrangement of O-rings 230 to accommodate the sealing of the upper tube 50 with the drum partitions 170.

The apparatus of the invention may require gas-tight seals in the adjustable accumulator 20 at the contact points between the adjacent detachable drum compartments 180. A seal between the adjacent detachable drum compartments 180 of the adjustable accumulator 20 can be achieved in several ways, depending on the material from which the adjustable accumulator 20 is fabricated. If the adjustable accumulator 20 is made of TEFLON™, the walls can be fabricated so that the contact between the TEFLON™ walls of adjacent detachable drum compartments forms a seal. If the adjustable accumulator 20 is made of metal, glass, or plastic, O-rings 230 or packing are generally needed to make a proper seal between the walls of adjacent detachable drum compartments 180. If O-rings 230 are to be used, FIGS. 17-19 show possible arrangements of O-rings 230 to accommodate the sealing of the drum compartments 180.

Preferably, the apparatus of the invention includes gas-tight seals at the junction between the first valve 30 and the reservoir 24, and between the second valve 32 and the sterilizer 10. Examples of gas-tight seals are the same as those mentioned previously for seals between walls of adjacent detachable drum compartments 180. Alternatively, if the valves are made out of glass, the portions of the valves can be made as a ground-glass joint to provide a gas-tight seal.

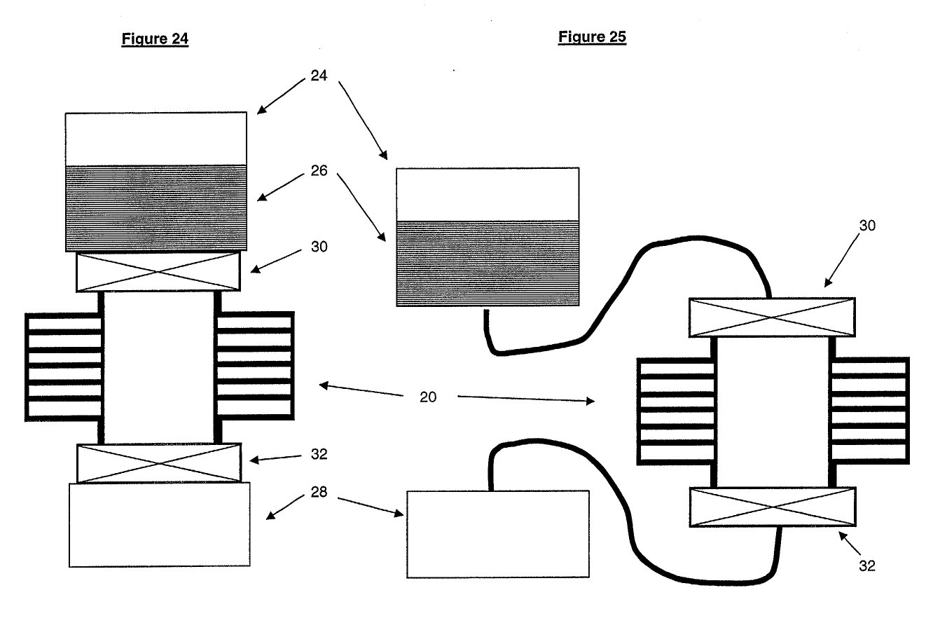

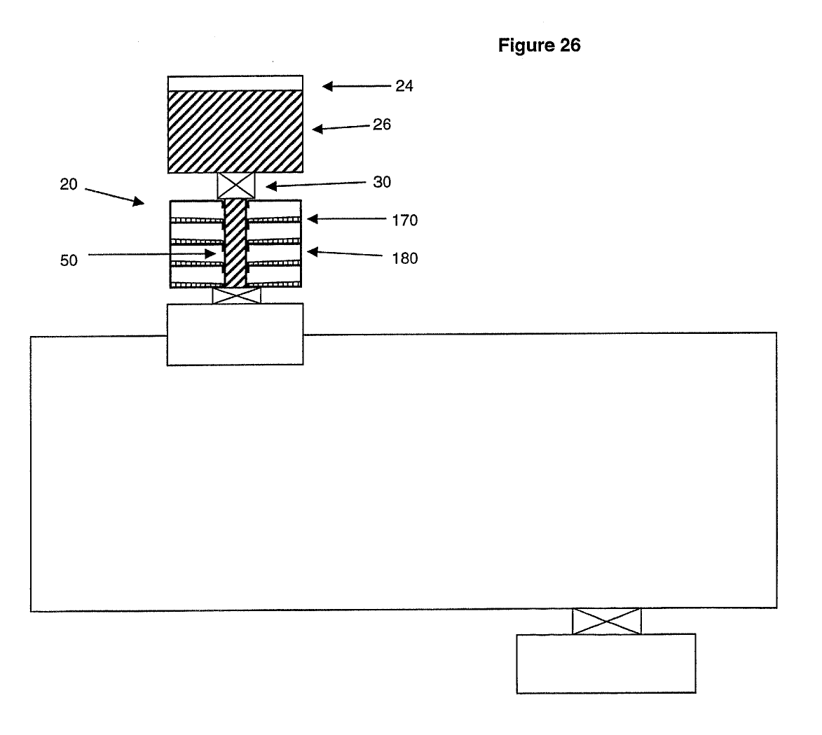

FIGS. 24-25 show different embodiments with regards to the accumulation of vaporizable germicide 26 in the adjustable accumulator 20. FIG. 24 shows the adjustable accumulator 20 that is connected below the reservoir 24 and above the vaporizer 28. The first valve 30 is opened to transfer the vaporizable germicide 26 from the reservoir 24 to the adjustable accumulator 20. Likewise, after the vaporizable germicide 26 has collected in the adjustable accumulator 20, the second valve 32 is opened to transfer the vaporizable germicide 26 from the adjustable accumulator 20 to the vaporizer 28. Both transfers of vaporizable germicide can be conducted with either gravity, pressure, or vacuum. In another embodiment shown in FIG. 25, tubes connect the adjustable accumulator 20 to the reservoir 24 and the vaporizer 28. The transfer of vaporizable germicide proceeds because of vacuum. With the second valve 32 opened and the first valve 30 closed, the vacuum pump evacuates the adjustable accumulator 20 and the vaporizer 28. Then, with the second valve 32 closed and the first valve 30 opened, the adjustable accumulator 20 is filled with vaporizable germicide 26 from the reservoir 24 via the vacuum that had been created. Finally, with the first valve 30 closed and the second valve 32 opened, the vaporizable germicide 26 is transferred from the adjustable accumulator 20 to the vaporizer 28.

Returning again to FIG. 1, the accumulation of vaporizable germicide in the adjustable accumulator will now be discussed by applying an embodiment that uses either the gravity-fill, pressure-fill, or vacuum-fill method; however, the adjustable accumulator comprises other embodiments and this example is not viewed as limiting. The vaporizable germicide 26 is placed in the reservoir 24 above the first valve 30. The vaporizable germicide 26 can be any liquid vaporizable germicide including aqueous hydrogen peroxide, peracetic acid, chlorine dioxide, ozone, or formaldehyde. In a preferred embodiment, the vaporizable germicide 26 comprises aqueous hydrogen peroxide. In another preferred embodiment, the vaporizable germicide 26 is approximately 20-60 wt % aqueous hydrogen peroxide.

As shown in FIG. 26, the first valve 30 between the reservoir 24 and the adjustable accumulator 20 is opened, allowing the vaporizable germicide 26 to enter the upper tube 50 of the adjustable accumulator 20. The upper tube 50 is positioned at the lowest position. All drum partitions 170 fit tightly against the upper tube 50 and none of the drum compartments 180 is in fluid communication with the interior of the upper tube 50. As a result, the vaporizable germicide 26 flows into the upper tube 50 alone.

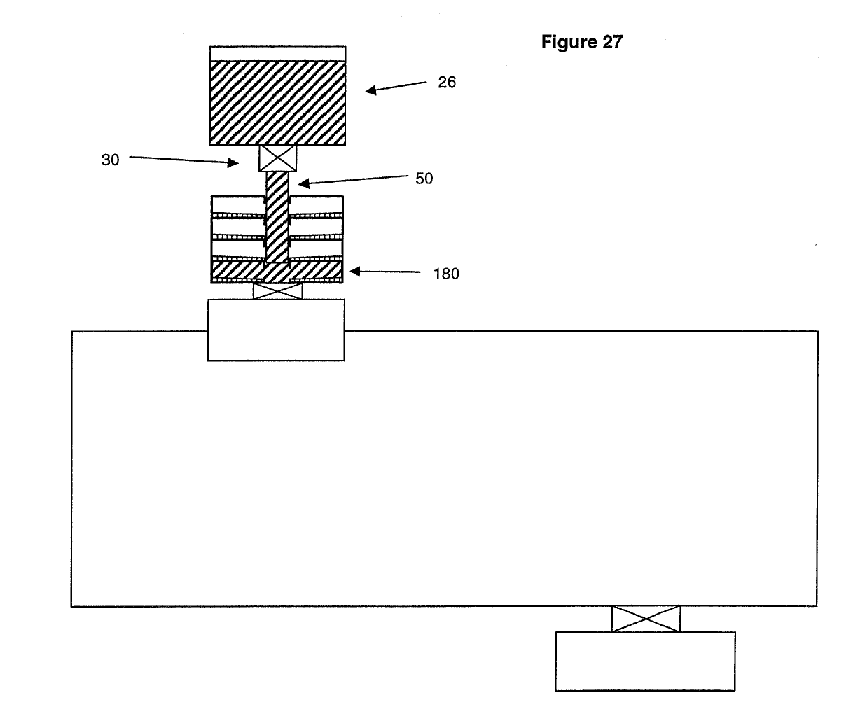

In FIG. 27, the upper tube 50 has been raised to expose the lowest drum compartment 180 into fluid communication with the interior of the upper tube 50, allowing vaporizable germicide 26 to enter the lowest drum compartment 180 upon opening the first valve 30. The drum compartment 180 provides additional volume to the upper tube 50 and allows delivery of a larger volume of the vaporizable germicide 26 than the upper tube 50 alone. The upper tube 50 can be held in place by the methods described previously.

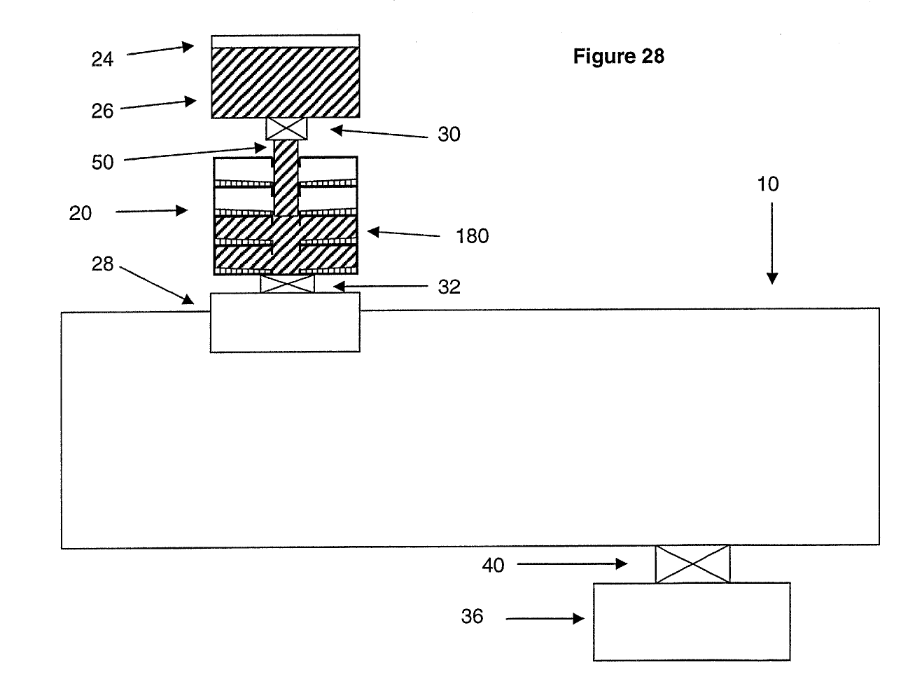

In FIG. 28, the upper tube 50 has been raised to expose the two lowest drum compartments 180 and allowing vaporizable germicide 26 to enter the two lowest drum compartment 180 upon opening the first valve 30. Raising the upper tube 50 further places additional drum compartments into fluid communication with the interior of the upper tube 50. Additional compartments provide even more volume of vaporizable germicide.

The volume of germicide contained in the adjustable accumulator can be varied by adjusting the level of the upper tube 50. The drum compartments 180 offer additional volume to the upper tube 50, when the drum compartments 180 are exposed to the interior of the upper tube 50. The drum compartments 180 allow delivery of a larger volume of germicide than the upper tube 50 alone. If the upper tube 50 is moved to a higher level, more drum compartments 180 are in fluid communication with the interior of the upper tube 50, thereby increasing the overall volume of vaporizable germicide available for delivery into the sterilization chamber. The adjustable accumulator allows for the delivery of a wide range of large volumes of sterilant to match the needs of various sizes of sterilization chambers. Consequently, the adjustable accumulator offers a simple, safe, and quick system to deliver sterilant to sterilization chambers.

After the desired amount of the vaporizable germicide 26 has accumulated in the adjustable accumulator 20, and the pressure of the sterilization chamber 10 reaches the desired pressure, the second valve 32 is then opened to transfer the vaporizable germicide 26 to the vaporizer 28. The vaporizer 28 is fluidly connected to the interior of the sterilization chamber 10. The vaporizer is preferably maintained at a temperature of approximately 50 to approximately 70o C. As the vaporizable germicide 26 enters the heated vaporizer 28, the vaporizable germicide 26 vaporizes, and the vapor enters the sterilization chamber 10. The germicide vapor contacts the equipment to be sterilized (not shown) in the sterilization chamber 10, sterilizing the equipment. Optionally, plasma is introduced into or is generated in the sterilization chamber 10 to enhance the sterilization by the germicide vapor or to remove residual germicide.

When the vaporizable germicide 26 comprises hydrogen peroxide and water, accumulating larger volumes of vaporizable germicide 26 in the adjustable accumulator 20 has advantages over allowing smaller amounts of vaporizable germicide 26 to enter the vaporizer 28. Water has a higher vapor pressure than hydrogen peroxide. If the valve 40 between the sterilization chamber 10 and the vacuum pump 36 is left open when the vaporizable germicide 26 is vaporizing in the vaporizer 28, water is preferentially removed from the sterilization chamber 10, because water has a higher vapor pressure than hydrogen peroxide, and the vapor in the sterilization chamber 10 is enriched in water vapor compared to the vaporizable germicide 26 in the vaporizer 28. Removing water from the aqueous hydrogen peroxide left in the vaporizer 28 by removing the water vapor in the sterilization chamber 10 concentrates the hydrogen peroxide in the vaporizer 28.

After a predetermined period of time, the valve 40 leading to the vacuum pump 36 is closed, and the concentrated hydrogen peroxide is allowed to vaporize from the vaporizer 28 into the sterilization chamber 10. The concentrated hydrogen peroxide in the vaporizer 28 vaporizes to produce a vapor which has a higher concentration of hydrogen peroxide than if water had not been removed from the aqueous hydrogen peroxide in the vaporizer 28 by preferential vaporization. The concentrated hydrogen peroxide vapor is more effective at sterilization than hydrogen peroxide vapor produced from a less concentrated solution of aqueous hydrogen peroxide.

With the use of the adjustable accumulator, the sterilization process is more effective and efficient. The adjustable accumulator provides a simple, safe, and precise system for introducing vaporizable germicide into a sterilization chamber in which the amount of vaporizable germicide can be varied. The adjustable accumulator can deliver a wide range of volumes of vaporizable germicide to match the needs of various sizes of sterilization chambers. When large amounts of hydrogen peroxide as the vaporizable germicide is administered to the sterilization chamber, water can be preferentially removed from the system under vacuum; this scenario portrays a more effective sterilization process because the vapor in the sterilization chamber is enriched with hydrogen peroxide.

Various modifications and alterations of this invention will be apparent to those skilled in the art without departing from the scope and spirit of this invention. It should be understood that the invention is not limited to the embodiments disclosed therein, and that the claims should be interpreted as broadly as the prior art allows.

What is claimed is:

1. An adjustable accumulator having a volume for delivering a chemical, said adjustable accumulator comprising:
a hollow outer tube having a first open end and a second end connected to a first valve; and
a hollow inner tube having a first open end and a second end connected to a second valve, said first open end of the inner tube adapted to movably and securely connect to the first open end of the outer tube so as to retain said chemical within said volume, said volume being the space enclosed within the outer tube, the inner tube, the first valve and the second valve.

2. The adjustable accumulator of claim 1, wherein the outer tube and the inner tube are cylindrical.

3. The adjustable accumulator of claim 1, wherein
the outer tube further comprises at least one drum extending from said outer tube; wherein
said drum has an interior; and wherein
said volume comprises said interior when said inner tube is adjusted to a position so that the interior of said drum is in fluid communication with said volume.

4. The adjustable accumulator of claim 3, wherein the at least one drum has a shape selected from the group consisting of oval, circular, and any of the polygons.

5. The adjustable accumulator of claim 3, wherein said drum does not surround the entire outer tube.

6. The adjustable accumulator of claim 3, wherein
said drum further comprises at least one partition to separate said interior of the drum into at least two compartments; and wherein
said compartment has a compartment interior and said volume comprises said compartment interior when said inner tube is adjusted to a position so that the compartment interior is in fluid communication with said volume.

7. The adjustable accumulator of claim 6, wherein at least one compartment is detachable from the adjustable accumulator.

8. The adjustable accumulator of claim 6, wherein at least two compartments are substantially identical in size and shape.

9. The adjustable accumulator of claim 6, wherein the partition is downwardly sloped toward to the inner tube.

10. The adjustable accumulator of claim 1, wherein said chemical comprises gas, liquid, solid, or any combination thereof.

11. The adjustable accumulator of claim 1, further comprising at least one O-ring to retain the chemical in the adjustable accumulator.

12. The adjustable accumulator of claim 1, further comprising at least one adjustable retainer that holds the inner tube and the outer tube at a desired position.

13. The adjustable accumulator of claim 12, wherein the retainer is a setscrew.

14. The adjustable accumulator of claim 12, wherein the retainer is a spacer.

15. The adjustable accumulator of claim 12, wherein the retainer is a shaft or jack.

16. A system for sterilizing equipment, said system comprising:
an adjustable accumulator according to claim 1, in which said chemical is a germicide;
a germicide reservoir in fluid communication with said adjustable accumulator so as to deliver said germicide to said adjustable accumulator; and
a sterilization chamber in fluid communication with said adjustable accumulator so as to receive said germicide from said adjustable accumulator.

17. The system of claim 16, wherein
the outer tube further comprises at least one drum extending from said outer tube; wherein
said drum has an interior; wherein
said volume comprises said interior when said inner tube is adjusted to a position so that the interior of said drum is in fluid communication with said volume.

18. The system of claim 17, wherein
said drum further comprises at least one partition to separate said interior of the drum into at least two compartments; wherein
said compartment has a compartment interior and said volume comprises said compartment interior when said inner tube is adjusted to a position so that the compartment interior is in fluid communication with said volume.

19. The system of claim 16, further comprising a vaporizer, wherein
said vaporizer is in fluid communication with said adjustable accumulator via said second valve and is in fluid communication with said sterilization chamber.

20. The system of claim 16, further comprising a vacuum pump in fluid communication with said sterilization chamber.

21. The system of claim 16, further comprising a source of plasma.

22. The system of claim 16, wherein said first valve is located between said adjustable accumulator and said sterilization chamber and said second valve is located between said adjustable accumulator and said reservoir.

23. The system of claim 16, wherein said germicide comprises hydrogen peroxide.

24. A method for sterilizing an article in a chamber, said method comprising:
placing said article in said chamber;
delivering a volume of germicide from a reservoir containing germicide to said chamber via an adjustable accumulator according to claim 1 in which chemical is said germicide; and
contacting said germicide with said article; thereby
sterilizing said article in said chamber.

25. The method of claim 24, further comprising evacuating said chamber.

26. The method of claim 24, further comprising vaporizing said germicide.

27. The method of claim 24, wherein
the outer tube further comprising at least one drum extending from said outer tube; wherein
said drum has an interior; and wherein
said volume comprises said interior when said inner tube is adjusted to a position so that the interior of said drum is in fluid communication with said volume.

28. The method of claim 24, wherein said drum further comprises at least one partition to separate said interior of the drum into at least two compartments; wherein
said compartment has a compartment interior and said volume comprises said compartment interior when said inner tube is adjusted to a position so that the interior of said compartment is in fluid communication with said volume.

29. The method of claim 24, wherein said germicide comprises hydrogen peroxide.

30. The method of claim 24, further comprising opening and/or closing said second valve between said adjustable accumulator and said reservoir.

31. The method of claim 24, further comprising opening and/or closing said first valve between said adjustable accumulator and said chamber.

32. The method of claim 24, further comprising moving said inner tube and/or said outer tube so as to adjust the volume of the adjustable accumulator.

33. The method of claim 26, wherein the vaporizing step comprises opening said second valve.
